(19) United States
(12) Patent Application Publication
(10) Pub. No.: US 2020/0396797 A1
(43) Pub. Date: Dec. 17, 2020

(54) WARMING DEVICE HAVING CONVECTIVE DEVICE AND CONDUCTIVE HEATER

(71) Applicant: 3M INNOVATIVE PROPERTIES COMPANY, St. Paul, MN (US)

(72) Inventors: Winston T. Tan, Plymouth, MN (US); Philip G. Dion, Blaine, MN (US); Melanie L. Collins, Minneapolis, MN (US); Maryam Mazloumpour, Portland, OR (US); Ann M. Gilman, Woodbury, MN (US)

(21) Appl. No.: 16/303,368

(22) PCT Filed: May 18, 2017
(86) PCT No.: PCT/US2017/033222

§ 371 Date: Nov. 20, 2018

Related U.S. Application Data
(60) Provisional application No. 62/343,412, filed on May 31, 2016.

(51) Int. Cl. H05B 1/02; A61F 7/00

(52) U.S. Cl. CPC H05B 1/0272; A61F 2007/0233; H05B 2203/006; A61F 7/007

(57) ABSTRACT

At least some aspects of the present disclosure direct to a device including a conductive heater and a convective device. The conductive heater includes a first conductive bus and a second conductive bus, a first set of electrodes electrically connected to the first conductive bus, a second set of electrodes electrically connected to the second conductive bus, wherein the first and the second set of electrodes are interdigitated, and a plurality of heater stripes comprising printed ink and electrically connected to the first and the second sets of electrodes. The convective device includes a pneumatic structure and an opening into the pneumatic structure connected to an inflation medium source, wherein at least part of the convective device is air permeable. The application is further directed to a warming device comprising a gown and said device, wherein the conductive heater and convective device are disposed on or integrated with the gown.

### TECHNICAL FIELD

The present disclosure relates to warming devices.

### SUMMARY

At least some aspects of the present disclosure direct to a device comprising a conductive heater and a convective device. The conductive heater comprises a first conductive bus and a second conductive bus, a first set of electrodes electrically connected to the first conductive bus, a second set of electrodes electrically connected to the second conductive bus, the first and the second set of electrodes interdigitated, and a plurality of heater stripes comprising printed ink and electrically connected to the first and the second sets of electrodes. The convective device comprises a pneumatic structure and an opening into the pneumatic structure, wherein at least part of the convective device is air permeable.

At least some aspects of the present disclosure direct to a warming device, comprising a gown, a conductive heater and a convective device. The conductive heater comprises a first conductive bus and a second conductive bus, a first set of electrodes electrically connected to the first conductive bus, a second set of electrodes electrically connected to the second conductive bus, the first and the second set of electrodes interdigitated, and a plurality of heater stripes comprising printed ink and electrically connected to the first and the second sets of electrodes. The convective device comprises a pneumatic structure and an opening into the pneumatic structure, wherein at least part of the convective device is air permeable. The conductive heater and the convective device are disposed on or integrated with the gown.

### BRIEF DESCRIPTION OF DRAWINGS

The accompanying drawings are incorporated in and constitute a part of this specification and, together with the description, explain the advantages and principles of the invention. In the drawings,

FIG. 1A is a schematic view of an example of a conductive heater using printed ink;

FIG. 1B is a schematic view of another example of a conductive heater using printed ink;

FIG. 1C is a cross-sectional view of a conductive heater with some optional components;

FIG. 2A is an exploded view of one example of a warming device having a conductive heater and a convective device; FIG. 2B is a cross sectional view of the warming device illustrated in FIG. 2A;

FIG. 3 illustrates a gown having a conductive heater and a convective device; and

FIG. 4 shows a box diagram of a controller.

In the drawings, like reference numerals indicate like elements. While the above-identified drawing, which may not be drawn to scale, sets forth various embodiments of the present disclosure, other embodiments are also contemplated, as noted in the Detailed Description. In all cases, this disclosure describes the presently disclosed disclosure by way of representation of exemplary embodiments and not by express limitations. It should be understood that numerous other modifications and embodiments can be devised by those skilled in the art, which fall within the scope and spirit of this disclosure.

### DETAILED DESCRIPTION

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein. The use of numerical ranges by endpoints includes all numbers within that range (e.g. 1 to 5 includes 1, 1.5, 2, 2.75, 3, 3.80, 4, and 5) and any range within that range.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

At least some aspects of the present disclosure direct to a warming device having a conductive heater including electrodes and heater stripes producing heat and a convective device having a pneumatic structure. In some embodiments, the conductive heater can be battery powered. In some embodiments, the convective device can be connected to an inflatable medium source when in use. In some cases, the conductive heater and the convective device can be attached to each other and formed a layered structure. In some cases, the conductive heater and the convective device can be attached different portions of a garment such that they can be activated separately.

Convective devices generally refer to a device distributing matter in gas state. For example, convective devices can receive a stream of pressurized, warmed air, inflate in response to the pressurized air, distribute the warmed air within a pneumatic structure, and emit the warmed air onto a body to accomplish such objectives as increasing comfort, reducing shivering, and treating or preventing hypothermia. In some embodiments, a convective device has a pneumatic structure that is formed by two layers, each layer including one or more sheets, and at least one of the layers is air permeable that allows air distribution. As used herein, “inflatable” refers to a structure which increases in volume when air or other gas is supplied at a pressure greater than atmospheric pressure to the interior of the structure. Typically these structures inflate at relatively low pressures such as pressures less than 100 mmHg, preferably at pressures less than 50 mmHg, more preferably at pressures less than 25 mmHg. In some cases, the volume of the inflatable section can increase by greater than 100%.

At least some aspects of the present disclosure direct to a conductive heater having electrodes connected to power buses and printed ink heater stripes. In some embodiments, the conductive heater includes a conductive trace running parallel to the power bus. In such embodiments, the power bus may be formed with relative high resistance materials and low cost materials and the conductive trace are usually selected from relatively low resistance materials, such that the current distribution can be improved.

At least some aspects of the present disclosure direct to a warming device including a conductive heater and a convective device. In some embodiments, the conductive heater is powered by a battery. In some cases, the warming device in such configurations can be used for both pre-operation, during operation, and/or post operation. In some cases, the warming device in such configurations can provide heating in transit, for example, when it is powered by a battery. In some embodiments, the warming device can be integrated with or attached to a gown.

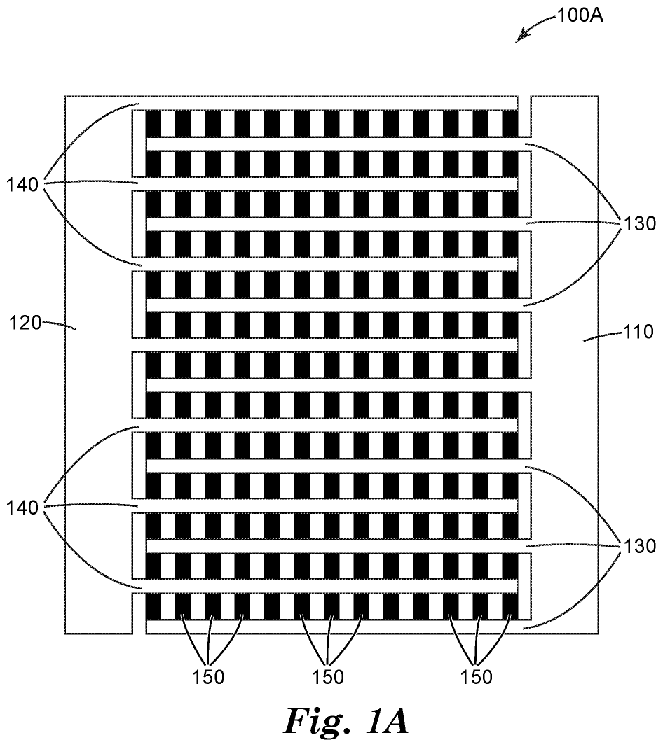

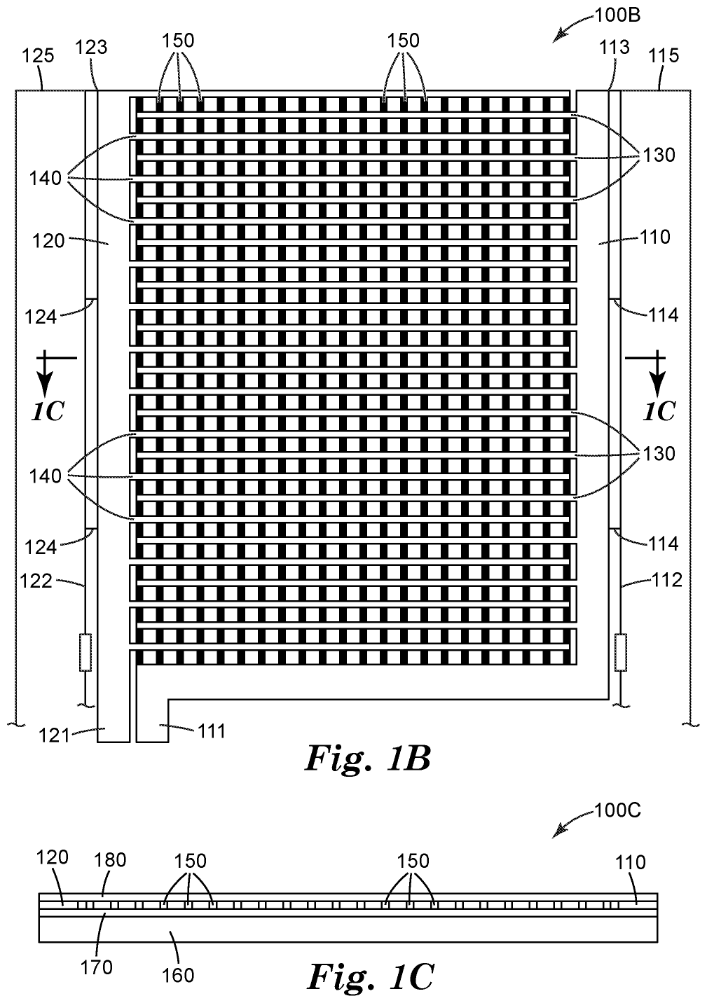

FIG. 1A is a schematic view of an example of a conductive heater 100A using printed ink. In some embodiments, the conductive heater 100A includes a first conductive bus 110, a second conductive bus 120, a first set of electrodes 130 electrically connected to the first conductive bus 110, a second set of electrodes 140 electrically connected to the second conductive bus 120, and a plurality of heater stripes 150 comprising printed ink and electrically connected to the first and the second sets of electrodes (130, 140). In some implementations, part of or all of the buses (110, 120), electrodes (130, 140), and heater stripes (150) can be screen printed.

In some embodiments, the printed ink can be positive temperature coefficient (PTC) ink. PTC ink has the property of self-regulating, where the resistance of ink starts to increase exponentially at a threshold temperature, such that the temperature of PTC ink can reach a maximum temperature. PTC ink can be, for example, PTC carbon ink or the like. In some embodiments, the PTC ink can be printed to cover the entire area of electrodes (130, 140). In some other embodiments, the PTC ink are printed in generally parallel strips 150 that are generally perpendicular to the electrodes (130, 140).

In some embodiments, the conductive bus (110, 120) can be generally parallel to each other. In some cases, the conductive bus (110, 120) can be designed with certain width to provide sufficient electrical power to the electrodes (130, 140). In the example as illustrated, the set of electrodes (130, 140) are generally perpendicular to the conductive buses (110, 120) and generally parallel to each other. In some cases, the first set of electrodes 130 and/or the second set of electrodes 140 are generally equal spacing between adjacent electrodes. In some cases, the first and the second set of electrodes (130, 140) are interdigitated. In some embodiments, the conductive buses and electrodes can include metals, for example, copper, silver, gold, aluminum, tinned-copper, platinum, or the like. In some cases, the conductive buses (110, 120) and the electrodes (130, 140) can use a same material. In some cases, the conductive buses (110, 120) can use a different material from the material of

FIG. 1B is a schematic view of another example of a conductive heater 100B using printed ink. In some embodiments, the conductive heater 100B includes a first conductive bus 110, a second conductive bus 120, a first conductive trace 112 connected to the first conductive bus at a first set of connection points 114, a second conductive trace 122 connected to the second conductive bus at a second set of connection points 124, a first set of electrodes 130 electrically connected to the first conductive bus 110, a second set of electrodes 140 electrically connected to the second conductive bus 120, and a plurality of heater stripes 150 comprising printed ink and electrically connected to the first and the second sets of electrodes (130, 140).

In some embodiments, the conductive traces (112, 122) can be metal wires, for example, copper wires. The conductive traces can include metals, for example, copper, silver, gold, aluminum, tinned-copper, platinum, or the like. In some cases, the conductive traces (112, 122) can be used to check the integrity of the conductive buses (110, 120). In one embodiment, the first conductive trace 112 is configured to generate a first signal at one of the first set of connection points 114, wherein the first signal is indicative to an electrical characteristic of the first bus 110. In some cases, the second conductive trace 122 is configured to generate a second signal at one of the second set of connection points 124, wherein the second signal is indicative to an electrical characteristic of the second bus 120. In some cases, the conductive traces (112, 122) can be overlaid with a conductive buses (110, 120) respectively. In some embodiments, the first set of connection points 114 and/or the second set of connection points 124 are generally equal spacing along the respective conductive trace, for example, 7.62 cm (3.0 inches) between adjacent connection points. In some embodiments, the first set of connection points 114 and/or the second set of connection points 124 has shorter distance between adjacent connection points when the connection points are further away from the power source.

The conductive heater 100 often requires carrying relatively large currents through the conductive bus to deliver heater current to the heater strips. If a conductive bus were to be compromised such as a crack or tear, the current flowing through the remaining part of the bus might develop a hot spot. To mitigate this potentially hazardous condition, sensing traces can be added to the conductive heater 100. In some embodiments, a sensing trace 115 can be connected to the first conductive bus 110 at a connection point 113. In some cases, a sensing trace 125 can be connected to the second conductive bus 120 at a connection point 123. In some embodiments, the sensing trace (115 and/or 125) is configured to generate a signal indicative to an electrical characteristic of the conductive bus.

In some implementations, the conductive buses (110, 120) are connected to a power source at connection points (111, 121). In some cases, the connection points (113, 123) of the sensing traces (115, 125) are away from the power source connection points (111, 121), for example, the power source connection points (111, 121) and the sensing trace connection points (113, 123) are at opposite ends of the connective buses (110, 120). In some cases, the sensing traces (115, 125) can be any form of a conductor, for example, a wire, a printed ink conductive trace, or the like. In some embodiments, the sensing traces measure the voltage at the connection point with the conductive bus. The voltage drop measured by the sensing trace(s) under normal conditions should be minimal. Any bus integrity error, which may interrupt current to generate heat, will create a greater voltage drop on the bus, for example, a voltage drop greater than a predetermined level. The voltage drop can be measured by any sensing circuit, for example, such as an analog-to-digital converter using a microcontroller, a comparator, or the like. In some implementations, a signal indicating bus integrity error may change the operation of the conductive heater, for example, to power off the heater.

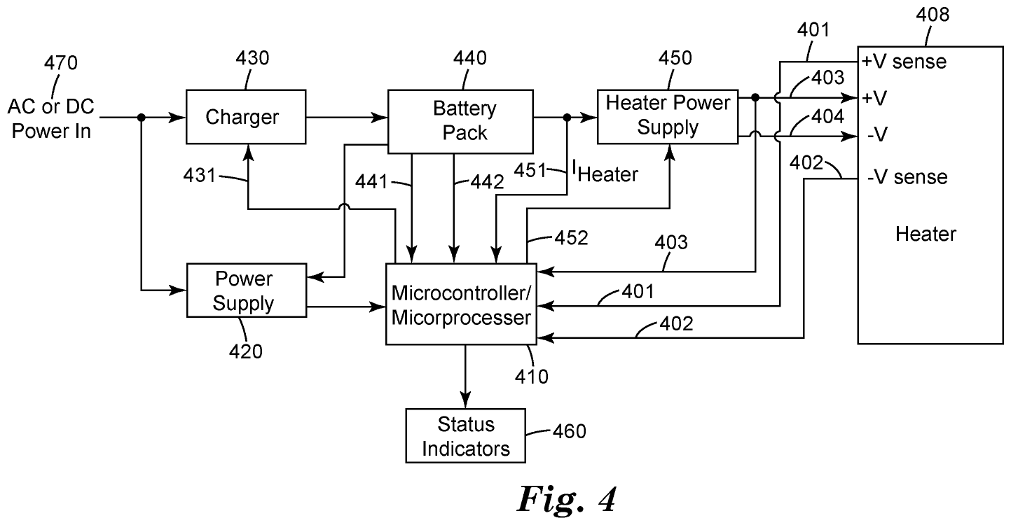

One example of a sensing circuit is provided in FIG. 4. One or more components of the sensing circuit can be optional. For example, the sensing circuit does not include some of the sensing signals or status indicators. In the example illustrated, the sensing circuit includes a conductive heater 400, a heater power supply 450, a microcontroller/microprocessor 410, a power supply 420, a charger 430, a battery pack 440, status indicators 460 and receives power from AC or DC power-in 470. The conductive heater 400 provides voltage sensing signal 401 and 402 and is powered by the heater power supply via signals 403 and 404. The charger 430 provides the required charging current and voltage to charge the battery pack 440. The charger 430 is controlled by the microcontroller 410 via a signal 431 to charge the battery with a profile that is appropriate for the battery chemistry that is chosen (e.g., Li-Ion). In some cases, the charger 430 is selected to have enough current capacity to both charge the batteries and power the heater when plugged in to main AC power.

In some embodiments, the battery pack 440 includes batteries that power the heater while the system is disconnected from the power-in 470. The size of the battery is determined by the desired amount of run time while the system is powered by batteries. Several battery parameters are monitored by the microcontroller 410. For example, a battery voltage 441 is monitored to provide a gas gauge indicator for the user, a charge control, and/or to allow low battery voltage shutdown to prevent battery damages. As another example, a battery temperature 442 is monitored to prevent overcharging damage and/or over current damage. In some cases, a heater current I_{Heater} 451 is monitored by the microcontroller 410 to measure how much current is being drawn from the battery. In response, the microcontroller 410 may control the heater powers supply 450 via a control signal 452.

The heater power supply 450 supplies the necessary voltage and current to drive the heater. These are determined by the heater design. For example, 24V DC with 4A of current capability can be used. The battery voltage can be higher or lower than the voltage supplied to the heater 400 via 403 and 404. In some cases, a DC-DC converter is used to step the battery voltage down or up to achieve the desired blanket voltage. The heater power supply 450 is controlled by the microcontroller 410 to turn on or off the heater. In some cases, the heater power supply 450 draws power off the battery pack 440 that supplies current from the batteries or from the power-in 470.

The power supply 420 supplies the required voltage and current to run the microcontroller circuit 410. In some embodiments, this is a low voltage, low current section that is always powered up. In some cases, the output voltage is in the 3V to 5V DC range depending on the microcontroller used. Current will typically be less than 100 mA. In some implementations, the power supply 420 draws power from either the power-in 470 or from the battery pack 440 if the power-in is disconnected.

In some embodiments, the microcontroller 410 controls the functions of the heater, provides safety monitoring, and/or provides status to the user. The microcontroller 410 is powered by the power supply 420. In some cases, the microcontroller 410 controls the charging of the battery pack 440, monitoring battery status (e.g., voltage, temperature, etc.), controlling the heater power supply 450, monitoring the heater status (e.g., heater resistance, heater current draw, conductive bus voltage drop, etc.), providing status to the user (e.g., heater on, battery level, AC present, charging, an indication of estimated run time before the battery is depleted. etc.) via the status indicators 460. The microcontroller 410 includes a variety of input/output interfaces to perform its functions including digital input/output lines for control, pulse width modulation for charger control, timers (e.g., for measuring power consumption and calculating battery status), digital-to-analog converters, and analog-to-digital converters for monitoring, for example, temperature, battery current draw, battery voltage, heater voltage, voltage drops on the conductive bus(es), and the like.

FIG. 1C is a cross-sectional view of a conductive heater 100C with some optional components. The conductive heater 100C can have same or similar elements, compositions, configurations and features as the corresponding components illustrated in FIGS. 1A-1B. Additionally, the conductive heater 100C can include an optional substrate 160. The substrate 160 can comprise a flexible, fibrous, preferably non-woven structure composed of polymeric materials, such as a non-woven, paper-based material, for example. In some embodiments, the conductive heater 100C can optionally include a layer of film 170. In some implementations, the plurality of heater stripes 150, the conductive buses (110, 120), and/or the electrodes (not illustrated) are disposed on the film 170. In some cases, the plurality of heater stripes 150, the conductive buses (110, 120), and/or the electrodes (not illustrated) can be printed on the film 170. The film 170 can has high surface tension, for example, using polyester, polyimide, glass-reinforced epoxy laminate sheet, or the like. In some cases, the conductive heater 100C can include an optional barrier film 180 disposed on the outer surface of the conductive heater 100C. The barrier film 180 can be a layer of dielectric material, for example, a polymeric film. In some embodiments, the barrier film can be formed by spray or silk-screen printing.

In some embodiments, the conductive heater 100C can be a portable device including a battery. In such embodiments, the battery can be connected to the first and second conductive bus to supply power to the heater. In some embodiments, the battery can use a rechargeable battery, for example, Li-Ion battery, Li-Iron battery, Ni-MH battery, Lead Acid battery, Ni—Cd battery, or the like. In some embodiments, the battery can use a non-rechargeable battery. In some cases, the conductive heater 100C can include a battery recharge circuit to recharge the battery.

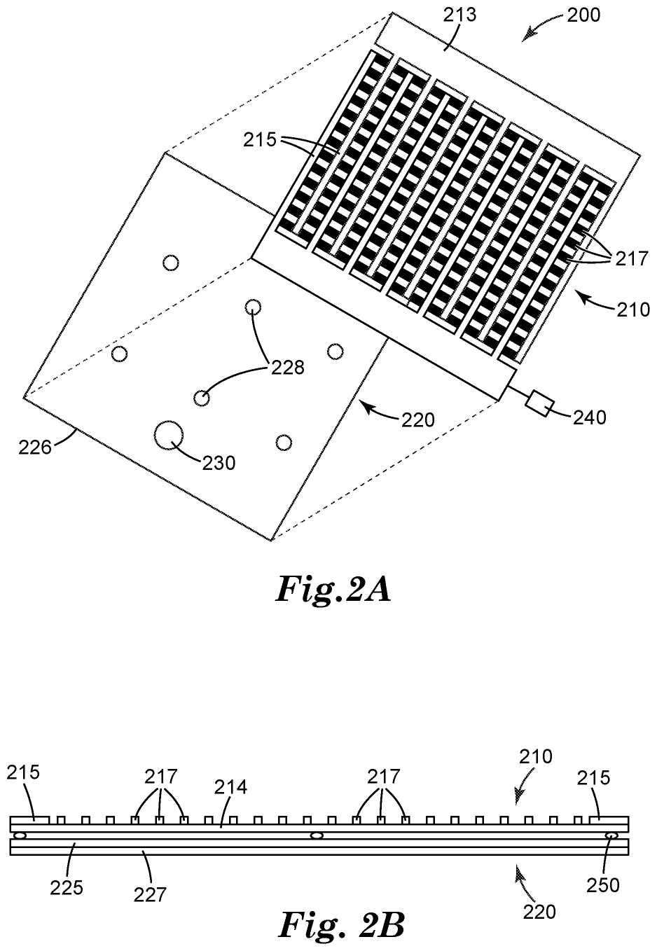

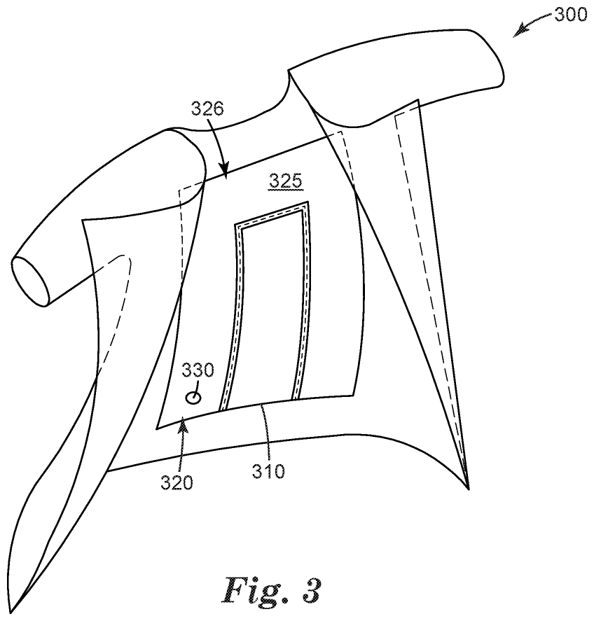

In some embodiments, a conductive heater can be used together with a convective device, such that, for example, the conductive heater powered by a battery can be used when the user is in transit. The conductive heater can use any of the designs and configurations described herein. FIG. 2A is an exploded view of one example of a warming device 200 having a conductive heater 210 and a convective device 220; and FIG. 2B is a cross sectional view of the warming device 200. The conductive heater 210 includes conductive buses 213, electrodes 215, heater stripes 217, and a substrate 214. In some cases, the conductive heater 210 is connected to a battery 240. In some implementations, the conductive buses 215 provide power to heater stripes 217 to generate heat. The convective device 220 includes a first layer 225 and a second layer 227 sealed at the peripheral to form an inflatable pneumatic structure.

Each of the first layer 225 and the second layer 227 may include one or more sheets, where each sheet may be formed from a different material. In some embodiments, the first layer 225 and/or the second layer 227 may include a sheet formed from a flexible, fibrous, for example, non-woven structure composed of polymeric materials. In some embodiments, the first layer 225 and/or the second layer 227 may include a sheet formed from a polymeric material including, for example, polyethylene, polyester, polypropylene (PP), high-density polyethylene (HDPE), polyethylene terephthalate (PET), polyamide (PA), or the like. In some implementations, the first layer 225 and/or the second layer 227 may include an underside sheet formed from a flexible, fibrous, preferably non-woven structure composed of polymeric materials capable of bonding to an upper side sheet of a heat-sealable polymeric material. For example, the underside sheet may be a non-woven, hydroentangled polyester material and the upper side sheet may include a polyolefin such as a polypropylene film which is extrusion-coated, thermally laminated, or adhesively laminated onto the polyester layer. Alternatively, the underside sheet may comprise a non-woven, paper-based material to which the upper side layer, including either a polyethylene or polypropylene film, has been glue laminated. In one embodiment, the upper side and underside sheets can be made with a stratum of absorbent tissue paper prelaminated with a layer of heat-sealable plastic. In some cases, both the first layer 225 and the second layer 227 can include a same polymer material.

In some embodiments, the second layer 227 includes the upper side sheet and the underside sheet, and the first layer 225 comprises the same material as the upper side sheet of the second layer 227. The first layer 225 thus may include a sheet of plastic bonded to the plastic upper side of the second layer 227. It is preferably attached by a continuously-running web process including stations that provide an interruptible heat-sealing process. This interruptible heat sealing process can be controlled to form elongated heat seals 228 that define the inflatable channels therebetween. The seals 228 can be formed as continuous air impervious seals or discontinuous air permeable seals. The interruptible heat sealing process can be used to form the continuous seams, one of which is the seam 226 at the peripheral of the second layer 227 and the first layer 225. In some cases, the interruptible heat sealing process can be used to form the discontinuous heat seals 228. In some embodiments, the heat seals 228 can have any shapes, for example, such as a circle, a rectangular, an elongated rectangular, a square, an oval, a triangle, a trapezium, a polygon, or the like. In some cases, absorbent material can be applied to the convective device 210, for example, applied as a single material layer. The absorbent material can be bonded to the upper plastic layer by heat processing or by adhesive bonding.

In some embodiments, the convective device 210 is enabled to bathe a patient in the thermally controlled inflation medium introduced into the convective device 210, when inflated, via an air permeable layer, the first layer 225 and/or the second layer 227. A layer can be air permeable using various materials or mechanical structures, for example, air-permeable materials, apertures, interstices, slits, or the like. In some implementations of an air permeable sheet with apertures, the density of apertures can vary among areas and/or inflatable sections.

In some embodiments, the first layer 225 and/or the second layer 227 are made from a polyolefin non-woven extrusion coated, each with a coating of polypropylene on one side. In some other embodiments, the first layer 225 and/or the second layer 227 can be poly lactic acid spunbond with polyolefin based extrusion coat. One of the first layer 225 and second layer 227 may have holes formed by punching, slitting, or cutting to permit the flow of pressurized inflation medium from the inflated section through the layer. In some cases, the holes can be opened through both layers. In some cases, when the convective device 210 is assembled, the polypropylene-coated side of the first layer 225 is sealed to the polypropylene-coated side of the second layer 227 at the periphery, and at the one or more locations to form the construction. The sealing process can use various techniques, for example, ultrasonic welding, radio frequency welding, heat sealing, or the like. Alternatively, the first layer 225 and second layer 227 may each include a laminate of polypropylene and polyolefin web with holes formed in at least one of the layers to support passage of pressurized air. In yet another embodiment, at least one of the layers can use air permeable material, for example, spunbond-meltblown-spunbond (SMS) nonwoven material, or the like.

In some embodiments, the convective device 210 includes at least one opening 230 into the pneumatic structure formed by the first layer 225 and the second layer 227. The opening 230 can be in any form that allows an inflation medium source (not illustrated) to connect and provide inflation medium to inflate the pneumatic structure, for example, a sleeve opening at the edge. As other examples, the opening 230 can include one or more inlet ports, cuffs, ports with a rigid collar, sleeve openings at the edge, or the like.

In some embodiments, the warming device 200 includes an attachment device 250 configured to attach the conductive heater 210 to the convective device 220. In some embodiments, the attachment device 250 can use a releasable or non-releasable attachment means, for example, two-sided adhesive, perforated tear-away tabs, hook and loop, snaps, rivets, repositionable adhesives, mechanical reclosable fasteners, or the like. In some cases, the conductive heater 210 may be detached from the convective device 220 after the conductive heater 210 is used. In some cases, the conductive heater 210 can be air permeable, for example, including mechanical structures such as apertures, slits, or interstices.

In some embodiments, a gown can include a warming device to provide heating to a user. FIG. 3 illustrates a gown 300 having a conductive heater 310 and a convective device 320. The conductive heater 310 can use any configuration of conductive heaters described herein. The conductive heater 310 and the co0nvective device 320 can attach to or integrated with the gown 300. The convective device 320 can be a same or similar to the convective device illustrated in FIGS. 2A and 2B. The convective device 320 may include an opening 330 to connect to an inflation medium source. In some cases, the conductive heater 310 may be powered by a battery and configured to generate heat by heater stripes. In some cases, the convective device 320 may include a first layer 325 sealed to the gown 300 or a second layer (not illustrated) at the peripheral 326 of the first layer 325 to form a pneumatic structure. In the embodiment illustrated in FIG. 3, the conductive heater 310 is disposed on a first location of the gown 300 and the convective device 320 is disposed on a second location of the gown 300 different from the first location. In some embodiments, the conductive heater 310 and/or the convective device 320 is attached to the gown 300 by an attachment device. In some embodiments, the attachment device can use a releasable or non-releasable attachment means, for example, two-sided adhesive, perforated tear-away tabs, hook and loop, snaps, rivets, repositionable adhesives, mechanical reclosable fasteners, or the like.

### Exemplary Embodiments

Item A1. A device, comprising:

a conductive heater comprising:
a first conductive bus and a second conductive bus,
a first set of electrodes electrically connected to the first conductive bus,
a second set of electrodes electrically connected to the second conductive bus, the first and the second set of electrodes interdigitated,
a plurality of heater stripes comprising printed ink and electrically connected to the first and the second sets of electrodes; and

a convective device comprising a pneumatic structure and an opening into the pneumatic structure, wherein at least part of the convective device is air permeable.

Item A2. The device of Item A1, further comprising:

an attachment device configured to attach the conductive heater to the convective device.

Item A3.The device of Item A2, wherein the attachment device is releasable.

Item A4. The device of any one of Item A1-A3, wherein the plurality of heater stripes are generally perpendicular to the first and second sets of electrodes.

Item A5. The device of any one of Item A1-A4, wherein the first set of electrodes are generally parallel to each other.

Item A6. The device of Item A3, wherein the first set of electrodes are generally equal spacing.

Item A7. The device of any one of Item A1-A6, wherein the second set of electrodes are generally parallel to each other.

Item A8. The device of Item A7, wherein the second set of electrodes are generally equal spacing.

Item A9. The device of any one of Item A1-A8, wherein the conductive heater further comprises a barrier layer disposed on an outer surface of the conductive heater.

Item A10. The device of any one of Item A1-A9, wherein the conductive heater further comprises a battery connected to the first and second conductive bus.

Item A11. The device of any one of Item A1-A10, wherein the printed ink comprises positive temperature coefficient ink.

Item A12. The device of any one of Item A1-A11, wherein the conductive heater further comprises a substrate comprising a layer of non-woven material.

Item A13. The device of Item A12, wherein the substrate further comprises a layer of film.

Item A14. The device of Item A13, wherein the plurality of heater stripes are disposed on the layer of film.

Item A15. The device of any one of Item A1-A14, wherein the attachment device comprises at least one of a two-sided adhesive, a perforated tear-away tab, a hook and loop, a snap, a rivet, a repositionable adhesive, a mechanical reclosable fastener.

Item A16. The device of any one of Item A1-A15, further comprising: a gown, wherein the conductive heater and the convective device are disposed on or integrated with the gown.

Item A17. The device of Item A16, wherein the conductive heater is disposed on a first location of the gown and the convective device is disposed on a second location of the gown different from the first location.

Item A18. The device of any one of Item A1-A17, wherein the device is disposable.

Item A19. A warming device, comprising:

a gown,

a conductive heater comprising:
a first conductive bus and a second conductive bus,
a first set of electrodes electrically connected to the first conductive bus,
a second set of electrodes electrically connected to the second conductive bus, the first and the second set of electrodes interdigitated,
a plurality of heater stripes comprising printed ink and electrically connected to the first and the second sets of electrodes;

a convective device comprising a pneumatic structure and an opening into the pneumatic structure, wherein at least part of the convective device is air permeable,

wherein the conductive heater and the convective device are disposed on or integrated with the gown.

Item A20. The warming device of Item A19, wherein the warming device is disposable.

Item A21. The warming device of Item A 19 or A20, wherein the conductive heater is disposed on a first location of the gown and the convective device is disposed on a second location of the gown different from the first location.

Item A22. The warming device of any one of Item A19-A21, further comprising: an attachment device configured to attach the conductive heater to the gown.

Item A23. The warming device of Item A22, wherein the attachment device is releasable.

Item A24. The warming device of Item A22, wherein the attachment device comprises at least one of a two-sided adhesive, a perforated tear-away tab, a hook and loop, a snap, a rivet, a repositionable adhesive, a mechanical reclosable fastener.

Item A25. The warming device of any one of Item A19-A24, wherein the plurality of heater stripes are generally perpendicular to the first and second sets of electrodes.

Item A26. The warming device of any one of Item A19-A25, wherein the first set of electrodes are generally parallel to each other.

Item A27. The warming device of Item A26, wherein the first set of electrodes are generally equal spacing.

Item A28. The warming device of any one of Item A19-A27, wherein the second set of electrodes are generally parallel to each other.

Item A29. The warming device of Item A28, wherein the second set of electrodes are generally equal spacing.

Item A30. The warming device of any one of Item A19-A29, wherein the conductive heater further comprises a barrier layer disposed on an outer surface of the conductive heater.

Item A31. The warming device of any one of Item A19-A30, wherein the conductive heater further comprises a battery connected to the first and second conductive bus.

Item A32. The warming device of any one of Item A19-A31, wherein the printed ink comprises positive temperature coefficient ink.

Item A33. The warming device of any one of Item A19-A32, wherein the conductive heater further comprises a substrate.

Item A34. The warming device of Item A33, wherein the substrate further comprises a layer of non-woven material.

Item A35. The warming device of Item A33, wherein the substrate further comprises a layer of film.

Item A36. The warming device of Item A35, wherein the plurality of heater stripes are disposed on the layer of film.

Item B 1. A heater, comprising:

a first conductive bus and a second conductive bus,

a first set of electrodes electrically connected to the first conductive bus,

a second set of electrodes electrically connected to the second conductive bus, the first and the second set of electrodes interdigitated,

a plurality of heater stripes comprising printed ink and electrically connected to the first and the second sets of electrodes,

a first conductive trace connected to the first conductive bus at a first set of connection points, and

a second conductive trace connected to the second conductive bus at a second set of connection points.

Item B2. The heater of Item B1, further comprising: a sensing trace connected to the first conductive bus at a first sensing connection point.

Item B3. The heater of Item B2, wherein the sensing trace is configured to generate a first signal indicative to an electrical characteristic of the first conductive bus.

Item B4. The heater of any one of Item B1-B3, wherein the first set of connection points are distributed generally equal spacing.

Item B5. The heater of any one of Item B1-B4, wherein the first set of connection points comprises one or more connection points.

Item B6. The heater of any one of Item B1-B5, wherein a distance two adjacent connection points of the first set of connection points is no less than three inches.

Item B7. The heater of any one of Item B1-B6, wherein the first conductive trace is overlaid on the first conductive bus.

Item B8. The heater of any one of Item B1-B7, wherein the plurality of heater stripes are generally perpendicular to the first and second sets of electrodes.

Item B9. The heater of any one of Item B1-B8, wherein the first set of electrodes are generally parallel to each other.

Item B10. The heater of any one of Item B1-B9, wherein the first set of electrodes are generally equal spacing.

Item B11. The heater of any one of Item B1-B10, wherein the second set of electrodes are generally parallel to each other.

Item B12. The heater of any one of Item B1-B11, wherein the second set of electrodes are generally equal spacing.

Item B13. The heater of any one of Item B1-B12, further comprising: a barrier layer disposed on an outer surface of the conductive heater.

Item B14. The heater of any one of Item B1-B13, further comprising: a battery connected to the first and second conductive bus.

Item B15. The heater of any one of Item B1-B14, wherein the printed ink comprises positive temperature coefficient ink.

Item B16. The heater of any one of Item B1-B15, further comprising: a substrate comprising a layer of non-woven material.

Item B17. The heater of Item B16, wherein the substrate further comprises a layer of film.

Item B18. The heater of Item B17, wherein the plurality of heater stripes are disposed on the layer of film.

Item B19. A heater, comprising:

a first conductive bus and a second conductive bus,

a first set of electrodes electrically connected to the first conductive bus,

a second set of electrodes electrically connected to the second conductive bus, the first and the second set of electrodes interdigitated,

a plurality of heater stripes comprising printed ink and electrically connected to the first and the second sets of electrodes,

a first conductive trace connected to the first conductive bus at a first set of connection points,

a second conductive trace connected to the second conductive bus at a second set of connection points, and

a first sensing trace connected to the first conductive bus at a first sensing connection point.

Item B20. The heater of Item B19, wherein the sensing trace is configured to generate a first signal indicative to an electrical characteristic of the first conductive bus.

Item B21. The heater of Item B19 or B20, further comprising: a second sensing trace connected to the second conductive bus at a second sensing connection point.

Item B22. The heater of any one of Item B19-B21, wherein the first set of connection points are distributed generally equal spacing.

Item B23. The heater of any one of Item B19-B22, wherein the first set of connection points comprises one or more connection points.

Item B24. The heater of any one of Item B19-B23, wherein a distance two adjacent connection points of the first set of connection points is no less than three inches.

Item B25. The heater of any one of Item B19-B24, wherein the first conductive trace is overlaid on the first conductive bus.

Item B26. The heater of any one of Item B19-B25, wherein the plurality of heater stripes are generally perpendicular to the first and second sets of electrodes.

Item B27. The heater of any one of Item B19-B26, wherein the first set of electrodes are generally parallel to each other.

Item B28. The heater of any one of Item B19-B27, wherein the first set of electrodes are generally equal spacing.

Item B29. The heater of any one of Item B19-B28, wherein the second set of electrodes are generally parallel to each other.

Item B30. The heater of any one of Item B19-B29, wherein the second set of electrodes are generally equal spacing.

Item B31. The heater of any one of Item B19-B30, further comprising: a barrier layer disposed on an outer surface of the conductive heater.

Item B32. The heater of any one of Item B19-B31, further comprising: a battery connected to the first and second conductive bus.

Item B33. The heater of any one of Item B19-B32, wherein the printed ink comprises positive temperature coefficient ink.

Item B34. The heater of any one of Item B19-B33, further comprising: a substrate comprising a layer of non-woven material.

Item B35. The heater of Item B34, wherein the substrate further comprises a layer of film.

Item B36. The heater of Item B35, wherein the plurality of heater stripes are disposed on the layer of film.

### EXAMPLES

TABLE 1
Component Materials

| Component | Product Number & Description | Source |
|---|---|---|
| PTC Ink | LOCTITE ECI 8045 E&C (Carbon Positive | Henkel Corporation North |
| | Temperature Coefficient (PTC) Ink, 45-48° C. | America, Rocky Hill, CT, USA |
| | self-regulating temperature) | |
| Silver Ink #1 | LOCTITE ECI 1010 Highly conductive, screen- | Henkel Corporation North |
| | printable, silver ink for PET film. | America, Rocky Hill, CT, USA |
| Silver Ink #2 | Dupont PE826 (Silver composite conductive ink | DuPont Microcircuit Materials |
| | for low voltage circuitry on flexible PET films) | Research Triangle Park, NC, USA |
| Dielectric | LOCTITE EDAG PF 455B E&C (Electrodag) | Henkel Corporation North |
| Coating | | America, Rocky Hill, CT, USA |
| Polyester (PET) | MELINEX 462, (2.0 mil, 50 micron) Clear | DuPont Teijin Films, Chester, |
| Film Substrate | Industrial Grade PET film. | VA, USA |
| Nonwoven | Polypropylene Nonwoven, SMS (spunbond- | First Quality Nonwovens Inc., |
| | meltblown-spunbond) | Hazel Township, PA, USA |
| Transfer Tape | 3M ™ Adhesive Transfer Tape 9472 | 3M Company, St. Paul, MN, USA |
| Copper Wire | Bare Copper Wire, 30 AWG, 0.010″ Diameter, | Commonly available |
| Patient Gown | BAIR PAWS Patient Warming Gown: Model 81003 | 3M Company, St. Paul, MN, USA |

### Example 1

A PTC heater assembly was prepared in the following manner. A sheet of the polyester film was cut to size, (30.5×30.5 cm). Silver Ink #1 was screen printed onto the PET film in a horizontal bar pattern as shown in FIGS. 1A-1C as electrodes 130. Next, the PTC ink was screen printed onto the PET film in a vertical bar pattern as shown FIGS. 1A-1C, as conductive stripes 150, orthogonally positioned over the top of the horizontal pattern of the silver ink electrodes 130. The inks were allowed to cure and dry in an oven at 80° C. Conductive bus bars 110 and 120, as shown in FIGS. 1A-1C, were created during the screen printing of the Silver Ink #1.

Sensing wires 115 and 125, as shown in FIG. 1B, were installed to each side of the bus bars to allow the integrity of the bus bars to be checked during heater operation. Sensing wires were made of 30 gauge bare copper wire.

A thin film of the dielectric coating, Henkel Loctite Electrodag PR-455B, was spray coated over the entirety of screen printed conductive ink (silver and PTC) patterns.

The double sided Transfer Tape film was used to laminate the PTC heater assembly between two similarly sized polypropylene SMS nonwoven layers on both sides of the PET film; both the side with the printed conductive ink patterns and the side without the printed conductive inks.

### Example 2

Example 2 was prepared in the same fashion as Example 1, except that Silver Ink #2 was used instead of Silver Ink #1. Additionally, a supplemental wire was needed for current flow for each bus bar and was installed as conductive traces 112 and 122, as shown in FIG. 2B. The supplemental wire was made of 30 gauge bare copper wire, which was the same type of wire, but in addition to the sensing wires.

### Example 3

A controller box was added to the heater assembly of Example 1. The controller box diagram is shown in FIG. 4. The controller controlled the subsections of the system, including: power control to the heater, battery charging, battery status, safety checks (over current, voltage anomalies, temperature anomalies, resistance checks, etc), user indicators, and user controls. The controller was comprised of discrete electronics: special function Integrated circuits, complex programmable integrated circuits (FPGA microcontroller). A DC-DC converter was used to generate the necessary drive voltage for the heater regardless of the battery voltage. A current sensing resistor was in line so that the heater power could be measured. The microcontroller used also measured the heater voltage and heater current thereby calculating heater power. Additionally the microcontroller controlled the DC-DC converter to control the heater power. The microcontroller monitored the battery voltage and ambient temperature (allowing it to approximate the needed heater power). A RS-232 port was present for diagnostics and test purposes. A 24 VDC lithium-ion battery was used to power the system.

### Example 4

The 24 VDC battery powered PTC heater assembly with microcontroller of Example 3 was mounted to a patient gown 3M™ Bair Paws™ Patient Warming Gown Model 81003 (available from 3M Company, St. Paul, Minn., USA) as shown in FIG. 3.

The present invention should not be considered limited to the particular examples and embodiments described above, as such embodiments are described in detail to facilitate explanation of various aspects of the invention. Rather the present invention should be understood to cover all aspects of the invention, including various modifications, equivalent processes, and alternative devices falling within the spirit and scope of the invention as defined by the appended claims and their equivalents.

1. A device, comprising:
a conductive heater comprising:
a first conductive bus and a second conductive bus,
a first set of electrodes electrically connected to the first conductive bus,
a second set of electrodes electrically connected to the second conductive bus, the first and the second set of electrodes interdigitated, and
a plurality of heater stripes comprising printed ink and electrically connected to the first and the second sets of electrodes; and
a convective device comprising an inflatable pneumatic structure and an opening into the inflatable pneumatic structure, wherein at least part of the convective device is air permeable.

2. The device of claim 1, further comprising:
an attachment device configured to attach the conductive heater to the convective device.

3. The device of claim 2, wherein the attachment device is releasable.

4. The device of claim 1, wherein the plurality of heater stripes are generally perpendicular to the first and second sets of electrodes.

5. The device of claim 1, wherein the first set of electrodes are generally parallel to each other.

6. The device of claim 1, wherein the printed ink comprises positive temperature coefficient ink.

7. The device of claim 1, wherein the conductive heater further comprises a substrate comprising a layer of non-woven material.

8. The device of claim 7, wherein the substrate further comprises a layer of film.

9. The device of claim 8, wherein the plurality of heater stripes are disposed on the layer of film.

10. The device of claim 1, further comprising: a gown,
wherein the conductive heater and the convective device are disposed on or integrated with the gown.

11. The device of claim 1, wherein the device is disposable.

12. A warming device, comprising:
a gown,
a conductive heater comprising:
a first conductive bus and a second conductive bus,
a first set of electrodes electrically connected to the first conductive bus,
a second set of electrodes electrically connected to the second conductive bus, the first and the second set of electrodes interdigitated, and
a plurality of heater stripes comprising printed ink and electrically connected to the first and the second sets of electrodes;
a convective device comprising a pneumatic structure and an opening into the pneumatic structure, wherein at least part of the convective device is air permeable;
wherein the conductive heater and the convective device are disposed on or integrated with the gown.

13. The warming device of claim 12, wherein the conductive heater is disposed on a first location of the gown and the convective device is disposed on a second location of the gown different from the first location.

14. The warming device of claim 12, further comprising:
an attachment device configured to attach the conductive heater to the gown.

15. The warming device of claim 12, wherein the printed ink comprises positive temperature coefficient ink.

16. The warming device of claim 1, wherein the inflatable pneumatic structure is formed by two layers, each layer including one or more sheets, and at least one of the layers is air permeable.

17. The warming device of claim 1, wherein the inflatable pneumatic structure increases in volume when air or other gas is supplied at a pressure greater than atmospheric pressure to an interior of the structure.
